(19) United States
(12) Patent Application Publication
(10) Pub. No.: US 2006/0258679 A1
(43) Pub. Date: Nov. 16, 2006

(54) Process of preparing ziprasidone mesylate

(76) Inventors: Alex Mainfeld, Herzilia (IL); Amir Gold, Herzelia (IL); Marioara Mendelovici, Rechovot (IL)

Correspondence Address: KENYON & KENYON LLP, ONE BROADWAY, NEW YORK, NY, 10004, US

(21) Appl. No.: 11/353,304

(22) Filed: Feb. 13, 2006

Related U.S. Application Data
(60) Provisional application No. 60/652,294, filed on Feb. 11, 2005.
(60) Provisional application No. 60/652,356, filed on Feb. 11, 2005.
(60) Provisional application No. 60/661,687, filed on Mar. 14, 2005.
(60) Provisional application No. 60/689,701, filed on Jun. 9, 2005.
(60) Provisional application No. 60/705,762, filed on Aug. 4, 2005.
(60) Provisional application No. 60/762,349, filed on Jan. 25, 2006.
(60) Provisional application No. 60/762,695, filed on Jan. 26, 2006.

(51) Int. Cl. A61K 31/496; C07D 417/14

(52) U.S. Cl. 514/254.04; 544/368

(57) ABSTRACT

The invention provides a process of preparing ziprasidone mesylate.

### RELATED APPLICATIONS

The present application claims the benefit of the following U.S. Provisional Patent Application No. 60/652,294 filed Feb. 11, 2005, 60/65,2356 filed Feb. 11, 2005, 60/661,687 filed Mar. 14, 2005, 60/689,701 filed Jun. 9, 2005, 60/705,762 filed Aug. 4, 2005, 60/762,349 filed Jan. 25, 2006 and 60/762,695 filed Jan. 26, 2006, all of which are herein incorporated by reference.

### FIELD OF THE INVENTION

The present invention is directed to a process for preparing crystalline and amorphous forms of ziprasidone comprising the step of spray drying a solution of ziprasidone mesylate.

### BACKGROUND OF THE INVENTION

Ziprasidone is an antipsychotic agent and is therefore useful for treating various disorders including schizophrenia, anxiety and migraine pain. Ziprasidone has the following structure:

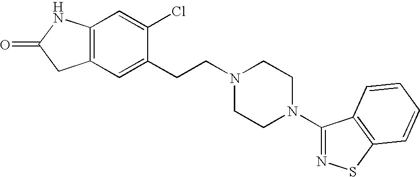

Ziprasidone is marketed under the name GEODON as an oral capsule and as an injectable drug. GEODON capsules contain the monohydrate hydrochloride salt of ziprasidone, and come in 20, 40, 60 and 80 mg dosage forms. GEODON for injection contains a lyophilized form of ziprasidone mesylate trihydrate, and contains 20 mg base equivalent of ziprasidone.

The present invention relates to the solid state physical properties of ziprasidone mesylate. These properties may be influenced by controlling the conditions under which ziprasidone mesylate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and may be used to distinguish some forms from others. A particular form may also give rise to distinct spectroscopic properties that may be detectable by powder X-ray crystallography, solid state C NMR spectrometry and infrared spectrometry.

The preparation of ziprasidone base is disclosed in U.S. Pat. No. 4,831,031 (example 16). Preparation of ziprasidone base is also disclosed in U.S. Pat. No. 5,312,925. U.S. Pat. No. 6,245,765 discloses dihydrate crystalline salts of ziprasidone mesylate and their use as dopamine antagonists. U.S. Pat. No. 6,110,918 discloses that four known ziprasidone mesylate crystalline forms exist. Each crystal form may be characterized by a distinct X-ray powder diffraction pattern and a distinct crystal shape that can be observed by photomicrograph. U.S. Pat. No. 6,110,918 also reports that the ziprasidone mesylate dihydrate lath crystals and dihydrate needle crystals are relatively long and thin in contrast to the prism crystals of ziprasidone mesylate trihydrate. In an aqueous medium at ambient temperature, ziprasidone mesylate trihydrate is reported to be the most thermodynamically stable form of the four crystalline forms of ziprasidone mesylate. U.S. Pat. No. 6,399,777 discloses the preparation of ziprasidone mesylate anhydrous forms by slurrying ziprasidone base and methanesulfonic acid in isopropyl alcohol.

There is a need in the art for improved processes for preparing amorphous forms of ziprasidone mesylate and/or processes for crystalline forms of ziprasidone mesylate.

### SUMMARY OF THE INVENTION

In one embodiment, the present invention provides a process of preparing amorphous ziprasidone mesylate comprising the step of spray-drying a solution of ziprasidone mesylate in a solvent selected from a group consisting of: C₁-C₅ alcohols, C₂-C₈ ethers, glacial acetic acid and mixtures thereof with water, using an outlet temperature of above about 90° C. Preferably the inlet temperature is above the outlet temperature.

In another embodiment, the present invention provides a process of preparing ziprasidone mesylate crystal form characterized by X-ray powder diffraction peaks at 11.7, 17.3, 23.5, 24.2, and 25.2 degrees two-theta, ±0.2 degrees two-theta (herein defined as Form I) comprising the step of spray-drying a solution of ziprasidone mesylate in a solvent selected from a group consisting of: glacial acetic acid and mixtures thereof with C₂-C₈ ethers using an outlet temperature of above about 70° C., and collecting the obtained Form I. Preferably the inlet temperature is above the outlet temperature.

In another embodiment, the present invention provides a process of preparing ziprasidone mesylate crystal form characterized by X-ray powder diffraction peaks at 17.1, 18.7, 23.8, and 24.4 degrees two-theta, ±0.2 degrees two-theta (herein defined as Form VIII) comprising the step of spray-drying a solution of ziprasidone mesylate in C₁-C₅ alcohols and mixtures thereof with water using an outlet temperature of from about above 45° C. to about 70° C. Preferably the inlet temperature is above the outlet temperature.

### BRIEF DESCRIPTION OF THE FIGURES

FIG. 1 illustrates the x-ray diffraction pattern of amorphous ziprasidone mesylate.

FIG. 2 illustrates the DSC thermogram of amorphous ziprasidone mesylate.

FIG. 3 illustrates the TGA thermogram of amorphous ziprasidone mesylate.

FIG. 4 illustrates the x-ray diffraction pattern of a mixture of ziprasidone mesylate amorphous and Form I.

FIG. 5 illustrates the x-ray diffraction pattern of ziprasidone mesylate Form I.

FIG. 6 illustrates the DSC thermogram of ziprasidone mesylate Form I.

FIG. 7 illustrates the TGA thermogram of ziprasidone mesylate Form I.

FIG. 8 illustrates the x-ray diffraction pattern of ziprasidone mesylate Form VIII.

FIG. 9 illustrates the DSC thermogram of ziprasidone mesylate Form VIII.

FIG. 10 illustrates the TGA thermogram of ziprasidone mesylate Form VIII.

### DETAILED DESCRIPTION OF THE INVENTION

The term “spray drying” broadly refers to processes involving breaking up liquid mixtures into small droplets (atomization) and rapidly removing solvent from the mixture. In a typical spray-drying apparatus, there is a strong driving force for evaporation of solvent from the droplets, which may be provided by providing a heated drying gas. Spray-drying processes and equipment are described in Perry's Chemical Engineer's Handbook, pgs. 20-54 to 20-57 (Sixth Edition 1984).

Publication No. US 2004/0194338 discloses a multitude of dispersions containing amorphous drugs and polymers, prepared by spray drying. The technique of spray drying has been used to produce powders, including bulk chemicals in powdered form. According to Remington: The Science and Practice of Pharmacy, 19th Ed., vol. II, pg. 1627, spray drying consists of bringing together a highly dispersed liquid and a sufficient volume of hot air to produce evaporation and drying of the liquid droplets. By way of non-limiting example only, a typical spray-drying apparatus comprises a drying chamber, atomizing means for atomizing a solvent-containing feed into the drying chamber, a source of heated drying gas that flows into the drying chamber to remove solvent from the atomized-solvent-containing feed and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray-drying are separated from the drying gas and evaporated solvent, allowing the particles to be collected. A filter may also be used to separate and collect the particles produced by spray-drying. The process of the invention is not limited to the use of such drying apparatuses as described above.

Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The ziprasidone mesylate product produced by spray-drying may be recovered by techniques commonly used in the art, such as using a cyclone or a filter.

In one embodiment, the present invention provides a process of preparing amorphous ziprasidone mesylate comprising the step of spray-drying a solution of ziprasidone mesylate in a solvent selected from a group consisting of: C₁-C₅ alcohols, C₂-C₈ ethers, glacial acetic acid and mixtures thereof with water, using an outlet temperature of above 90° C. Preferably the inlet temperature is above the outlet temperature.

Preferably, dihydrate needle crystals of ziprasidone mesylate are used to form the solution.

The solvent used to make the ziprasidone mesylate solution is in an amount sufficient to dissolve the ziprasidone mesylate, and can be determined by one skilled in the art with little or no experimentation. Preferably, the solvent is used in an amount of about 25 L to about 100 L per kilogram of ziprasidone mesylate.

Preferably, the solvent is selected from the group consisting of: diethyl ether, tetrahydrofuran, methyl t-butyl ether, glacial acetic acid, ethanol and mixtures thereof with water. More preferably, the solvent is an ethanol/water mixture.

Preferably, the ethanol/water ratio is from about 50:50 to about 95:5 ethanol to water by volume.

Preferably, the outlet temperature is set to about 90° C.

Preferably, the inlet temperature is above about 90° C., more preferably, about 150° C.

Whenever glacial acetic acid is used as a solvent both ziprasidone mesylate amorphous form and Form I may be obtained and the amorphous ziprasidone mesylate is collected from the product collection vessel.

Preferably, the amorphous ziprasidone mesylate obtained contains less than about 10% crystalline materials. More preferably, it contains less than about 5% crystalline materials. Most preferably, it contains less than about 1% crystalline materials.

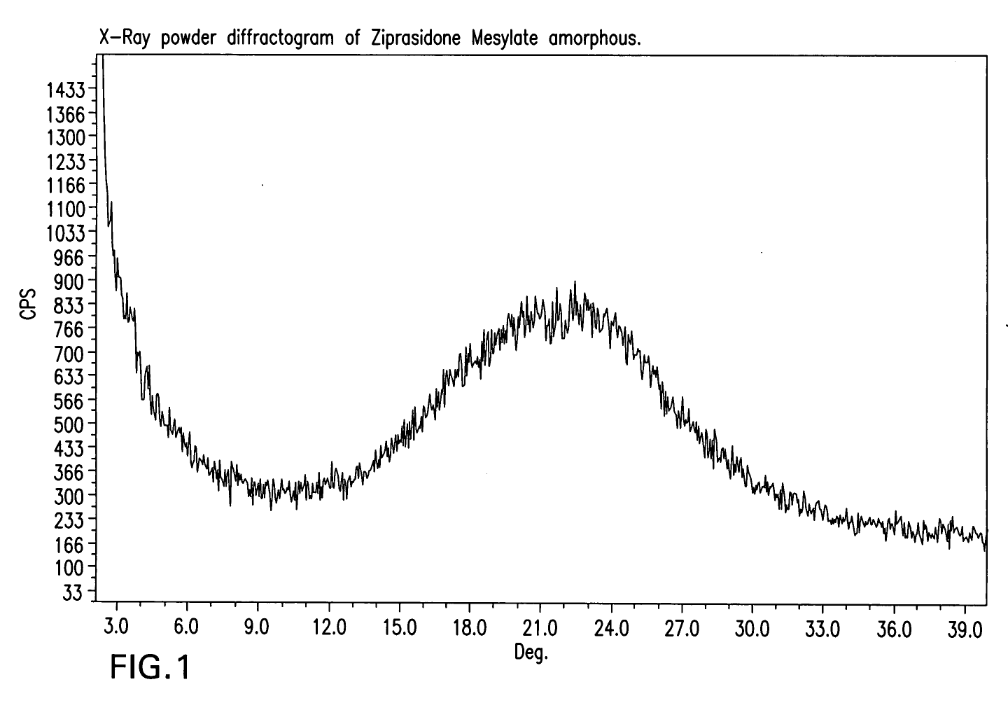

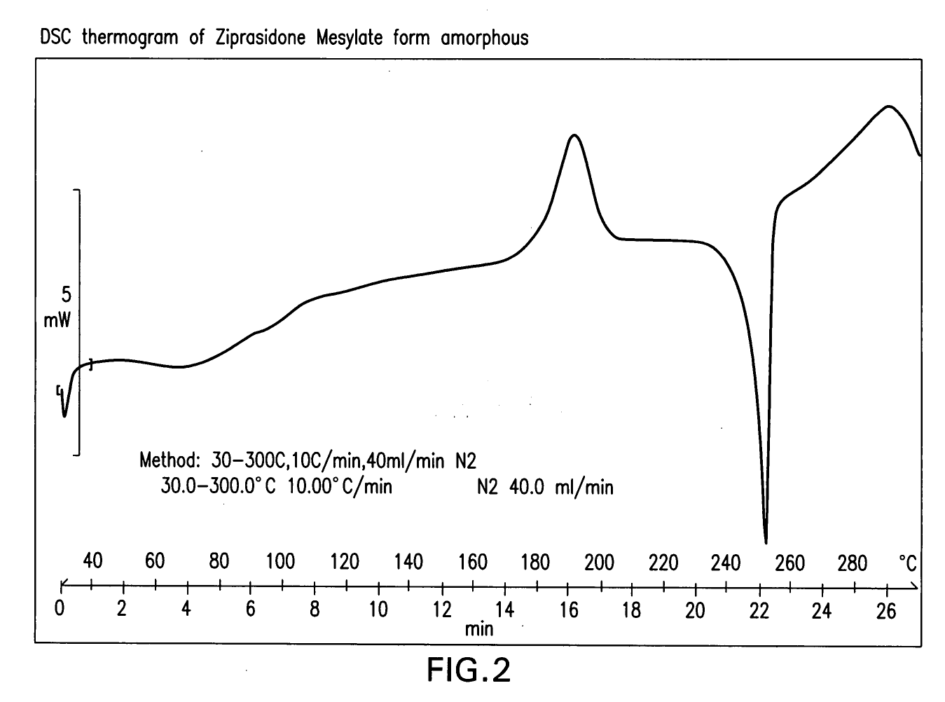

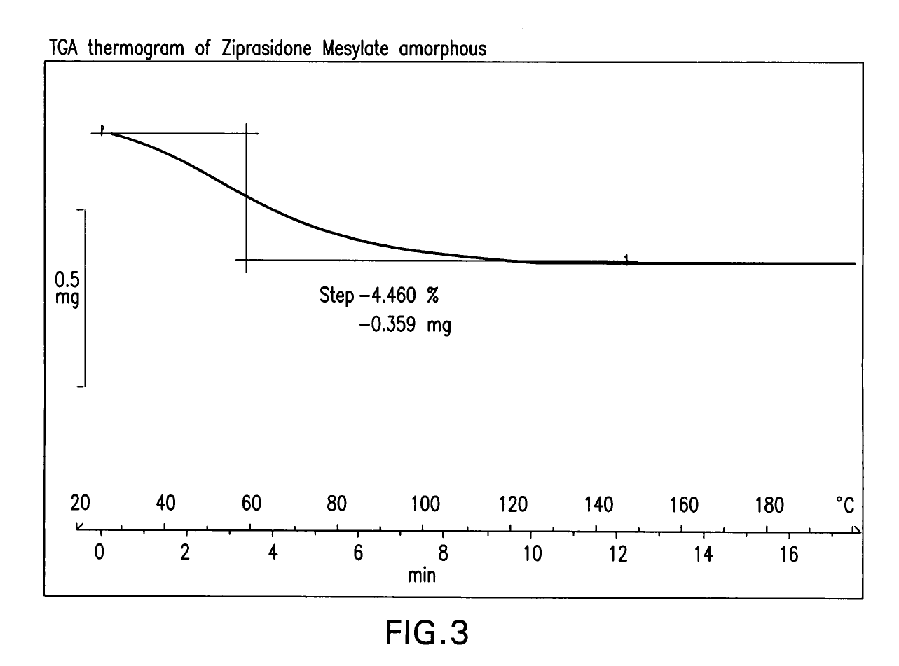

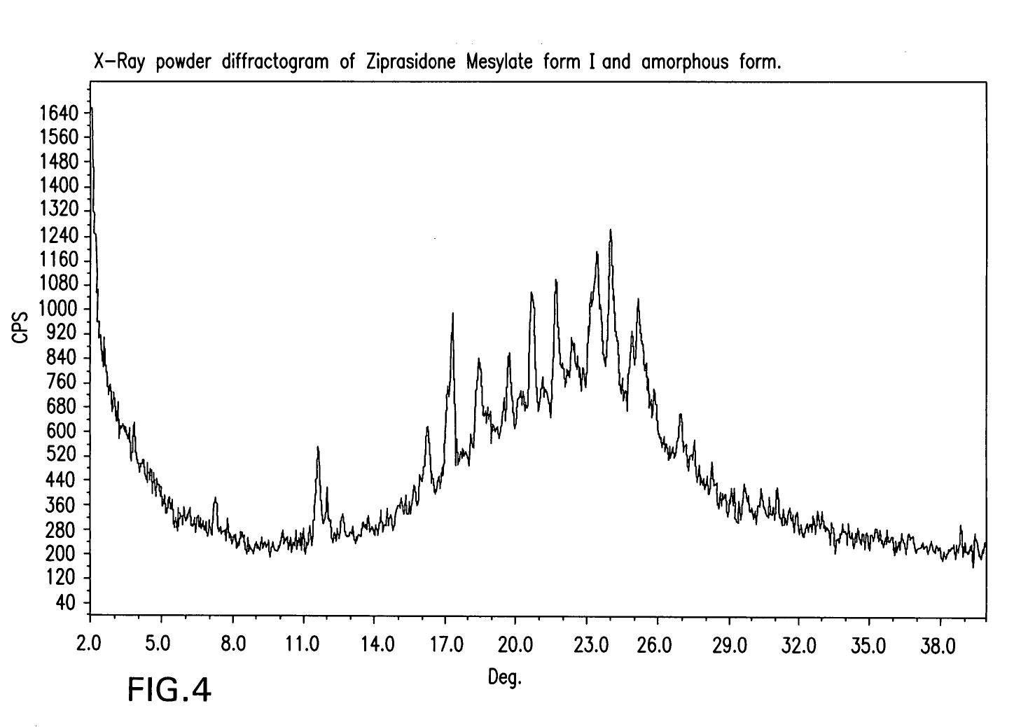

Amorphous form has an XRD pattern as substantially depicted in FIG. 1. Amorphous form also has a DSC thermogram and a TGA thermogram as substantially depicted in FIGS. 2 and 3, respectively. FIG. 4 substantially depicts the XRD pattern of a mixture of Form I and amorphous form. The amorphous form of ziprasidone mesylate is disclosed in U.S. application Ser. No. ______.

In another embodiment, the present invention provides a process of preparing ziprasidone mesylate crystal form characterized by X-ray powder diffraction peaks at 11.7, 17.3, 23.5, 24.2, and 25.2 degrees two-theta, ±0.2 degrees two-theta (herein defined as Form I) comprising the step of spray-drying a solution of ziprasidone mesylate in a solvent selected from a group consisting of: glacial acetic acid and mixtures thereof with C₂-C₈ ethers using an outlet temperature of above about 70° C. and collecting the obtained Form I. Preferably the inlet temperature is above the outlet temperature.

Preferably, dihydrate needle crystals of ziprasidone mesylate are used to form the solution.

Preferably, the solvent is glacial acetic acid.

The solvent used to make the ziprasidone mesylate solution is in an amount sufficient to dissolve the ziprasidone mesylate, and can be determined by one skilled in the art with little or no experimentation. Preferably, the solvent is used in an amount of about 25 L to about 100 L per kilogram of ziprasidone mesylate.

Preferably, the outlet temperature is from about 70° C. to about 100° C.

Whenever glacial acetic acid is used as a solvent, both ziprasidone mesylate amorphous form and Form I may be obtained and Form I is collected from the upper side of the apparatus. More preferably, Form I is collected from the upper side of the cyclone.

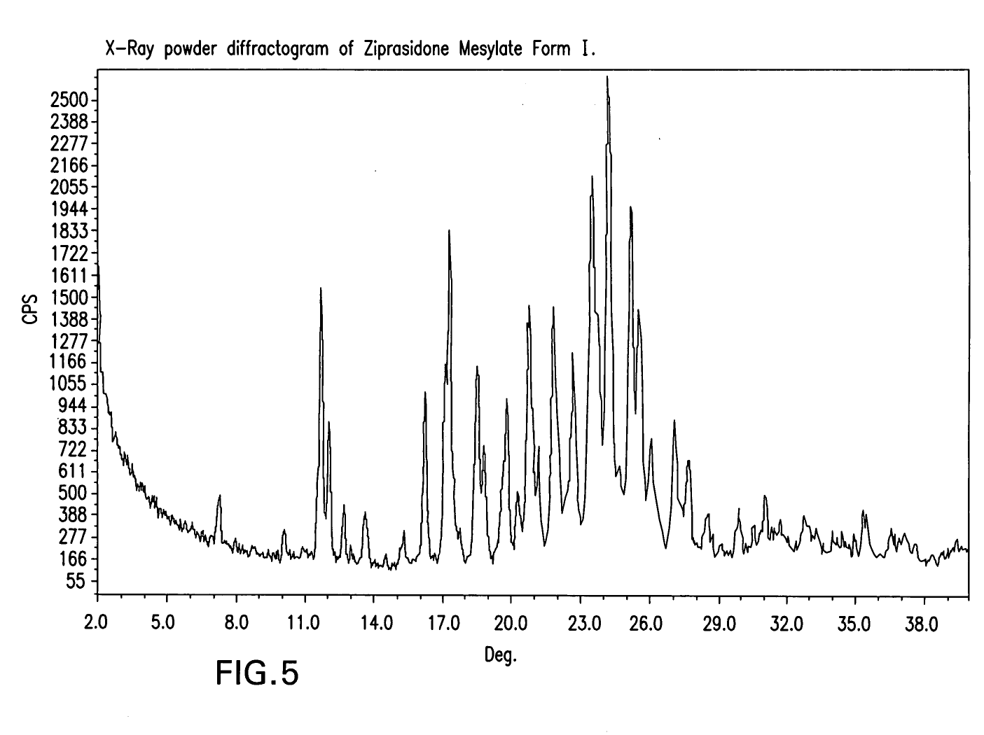

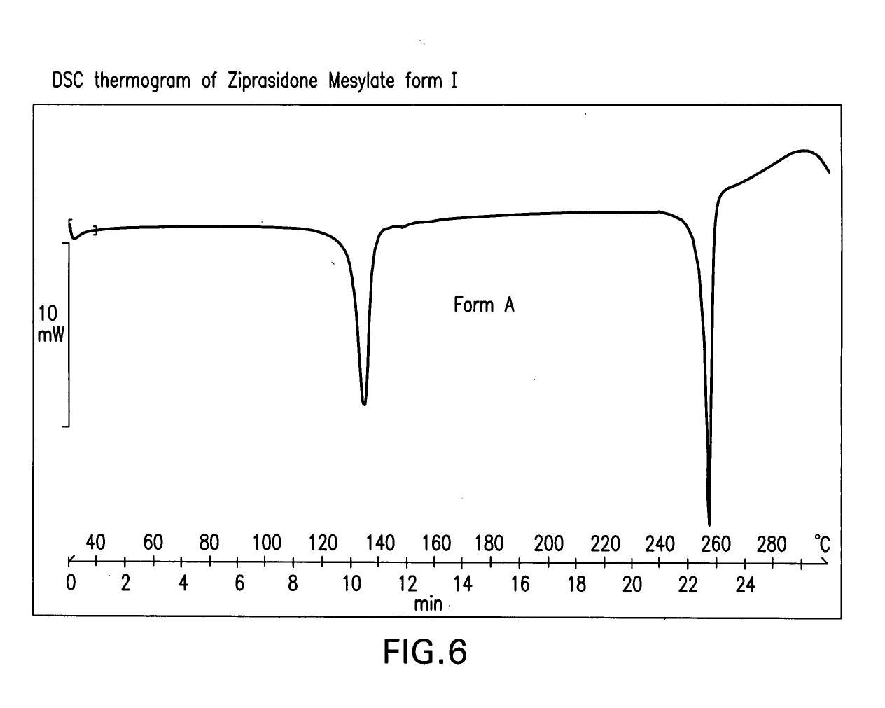

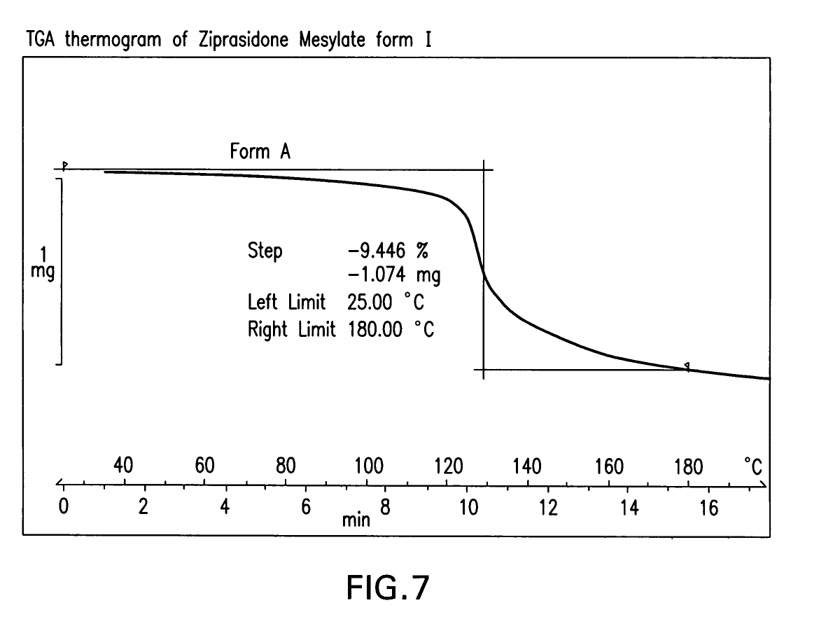

Form I may be further characterized by X-ray powder diffraction peaks at 18.5, 20.7, 21.8, 22.7, and 25.7 degrees two-theta, ±0.2 degrees two-theta. Form I has an XRD pattern as substantially depicted in FIG. 5. Form I also has a DSC thermogram and a TGA thermogram as substantially depicted in FIGS. 6 and 7, respectively. Form I of ziprasidone mesylate is disclosed in U.S. application Ser. No. ______.

In another embodiment, the present invention provides a process of preparing ziprasidone mesylate crystal form characterized by X-ray powder diffraction peaks at 17.1, 18.7, 23.8, and 24.4 degrees two-theta, ±0.2 degrees two-theta (herein defined as Form VIII) comprising the step of spray-drying a solution of ziprasidone mesylate in C₁-C₅ alcohols and mixtures thereof with water using an outlet temperature of from about above 45° C. to about 70° C. Preferably the inlet temperature is above the outlet temperature.

Preferably, dihydrate needle crystals of ziprasidone mesylate are used to form the solution.

Preferably, the solvent is a mixture of ethanol and water.

Preferably, the ethanol/water ratio is from about 50:50 to about 95:5 ethanol to water by volume.

The solvent used to make the ziprasidone mesylate solution is in an amount sufficient to dissolve the ziprasidone mesylate, and can be determined by one skilled in the art with little or no experimentation. Preferably, the solvent is used in an amount of about 25 L to about 100 L per kilogram of ziprasidone mesylate.

Preferably, the outlet temperature is set to about 55° C.

Preferably, the inlet temperature is above 55° C., more preferably, 80° C.

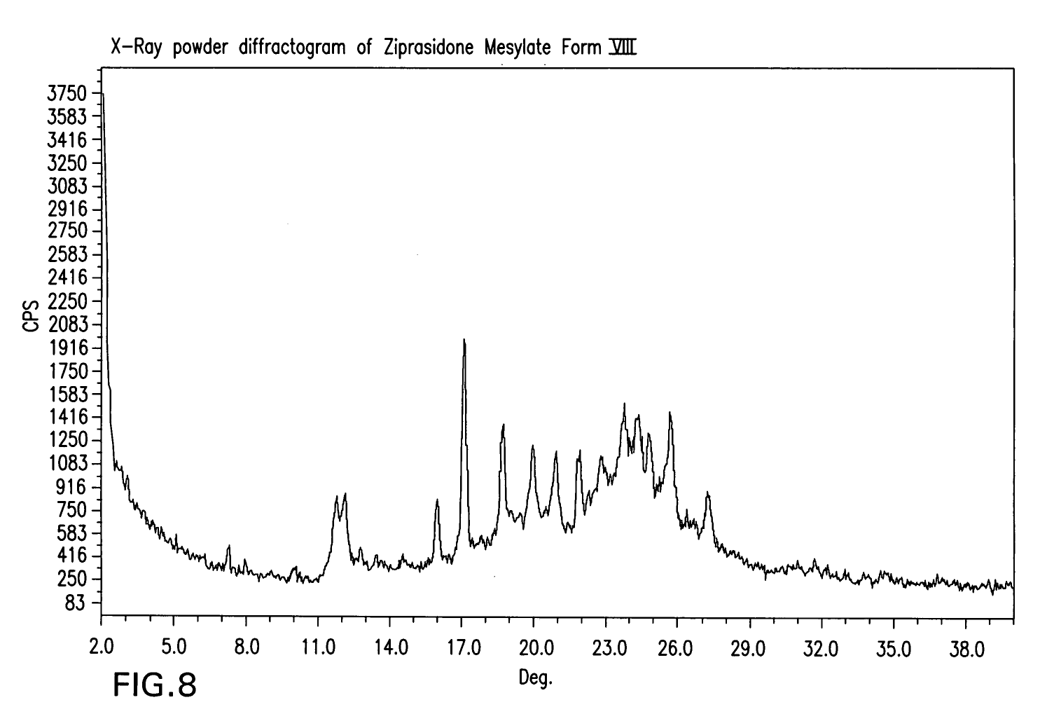

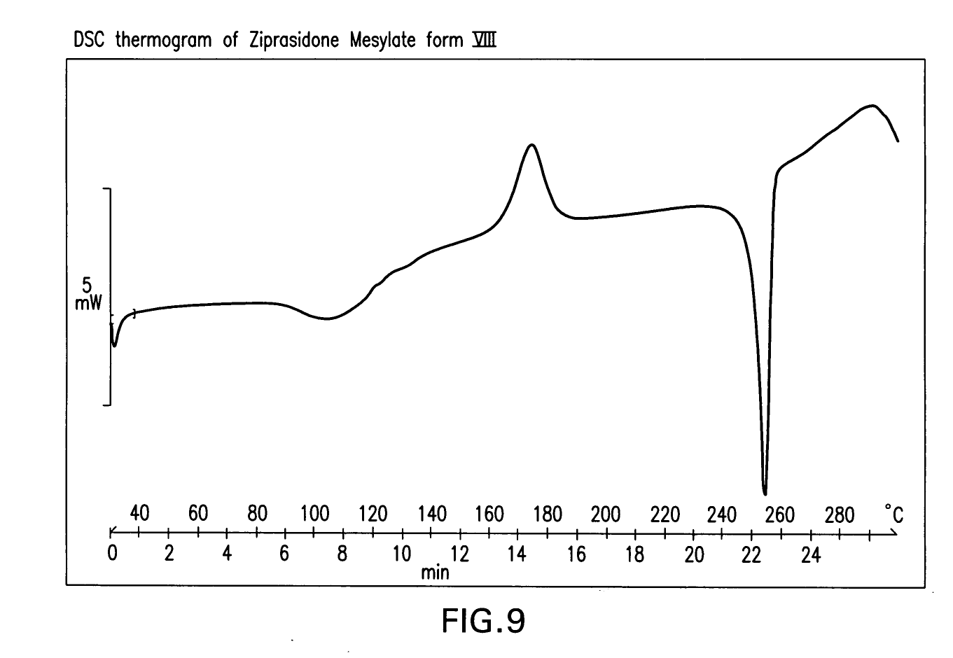

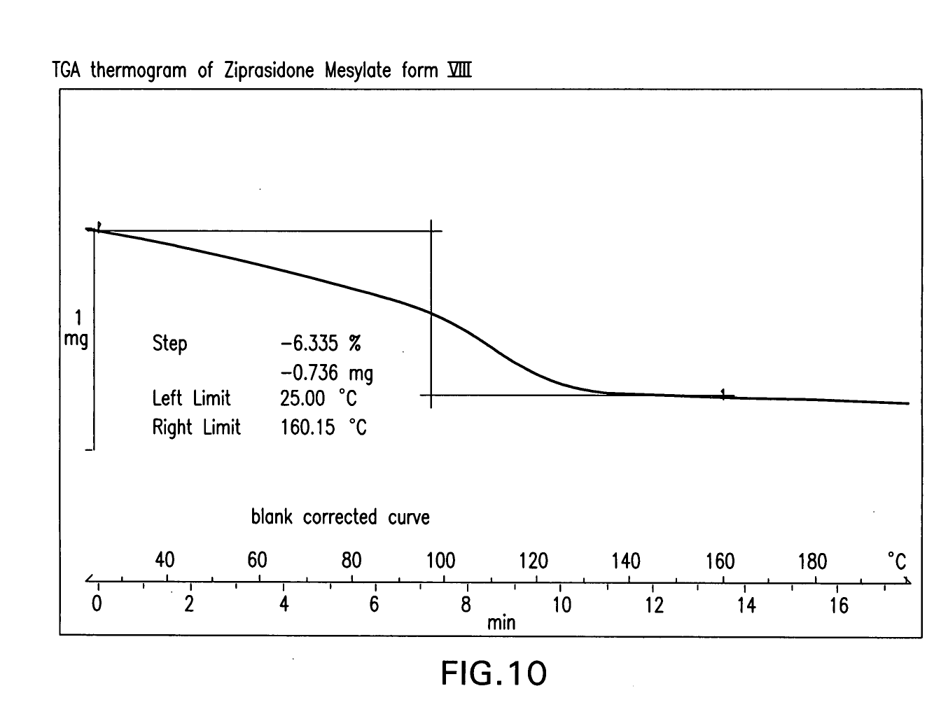

Form VIII may be further characterized by X-ray powder diffraction peaks at 11.8, 12.1, 20.0, 20.9, 24.9, and 25.7 degrees two-theta, ±0.2 degrees two-theta. Form VIII has an XRD pattern as substantially depicted in FIG. 8. Form VIII also has a DSC thermogram and a TGA thermogram as substantially depicted in FIGS. 9 and 10, respectively. Form VIII of ziprasidone mesylate is disclosed in U.S. application Ser. No. ______.

Having described the invention with reference to certain preferred embodiments, other embodiments will become apparent to one skilled in the art from consideration of the specification. The invention is further defined by reference to the following examples describing in detail the process of the invention. It will be apparent to those skilled in the art that many modifications, both to materials and methods, may be practiced without departing from the scope of the invention.

### EXAMPLES

Experimental

X-Ray powder diffraction data were obtained using a SCINTAG powder X-Ray diffractometer model X'TRA equipped with a solid state detector. Copper radiation of 1.5418 Å was used. A round aluminum sample holder with zero background was used. All peak positions are within +0.2 degrees two theta.

DSC analysis was performed using a Mettler 821 Stare. The weight of the samples is about 3-6 mg; the samples were scanned at a rate of 10° C./min from 30° C. to at least 300° C. The oven is constantly purged with nitrogen gas at a flow rate of 40 ml/min. Standard 40 μl aluminum crucibles covered by lids with 3 holes were used.

TGA analysis was performed using a Mettler M3 thermogravimeter. The weight of the samples is about 8 mg; the samples were scanned at a rate of 10° C./min from 25° C. to 200° C. A blank was subtracted from the sample. The oven is constantly purged with nitrogen gas at a flow rate of 40 ml/min. Standard 150 μl alumina crucibles covered by lids with 1 hole were used.

Karl Fisher analysis was performed according to the known art.

Microscope: The material was dispersed in a light mineral oil before the measurement.

### Example 1

### Preparation of Ziprasidone Mesylate Form I and Amorphous Ziprasidone Mesylate

Wet ziprasidone mesylate dihydrate needle crystals (15 g) were dissolved in glacial acetic acid (43 g). Using a Buchi Mini Spray Drier B-295 having a cyclone collection chamber, the ziprasidone mesylate solution was sprayed at a spray volume of 440 ml/hr into a chamber containing a parallel flow of nitrogen gas heated to about 100° C. (flow rate of about 38 m³/hr). The atomizing flow (6601/h) of nitrogen leads to a high evaporation rate. The outlet temperature was maintained at about 70° C.

A fraction was collected from the upper side of the cyclone, and determined to be ziprasidone mesylate Form I by XRD. The ziprasidone mesylate Form I collected contained a water content of about 2.3% by Karl Fisher analysis.

A fraction was collected from the lower side of the cyclone, and determined to be amorphous ziprasidone mesylate by XRD.

### Example 2

### Preparation of Amorphous Ziprasidone Mesylate

Wet ziprasidone mesylate dihydrate needle crystals (3.8 g) were dissolved in ethanol (80 ml) and water (20 ml). Using a Buchi Mini Spray Drier B-295 with an attached cyclone, the ziprasidone mesylate solution was sprayed at a spray volume of 440 ml/hr into a chamber containing a parallel flow of nitrogen heated to about 150° C. (flow rate of about 38 m3/hr). The atomizing flow (660 l/h) of nitrogen leads to a high evaporation rate. The outlet temperature was maintained at about 90° C. Amorphous ziprasidone mesylate, as determined by XRD, was collected from the cyclone, and had a water content of about 4.06% by Karl Fisher analysis.

### Example 3

### Preparation of Ziprasidone Mesylate Form VIII

Wet ziprasidone mesylate dihydrate needle crystals (5 g) were dissolved in ethanol (100 ml) and water (25 ml). Using a Buchi Mini Spray Drier B-295 with an attached cyclone, the ziprasidone mesylate solution was sprayed at a spray volume of 440 ml/hr into a chamber containing a parallel flow of nitrogen heated to about 80° C. (flow rate of about 38 m3/hr). The atomizing flow (660 l/h) of nitrogen leads to a high evaporation rate. The outlet temperature was maintained at about 55° C. Ziprasidone mesylate Form VIII, as determined by XRD, was collected from the cyclone, and had a water content of about 4% by Karl Fisher analysis.

What is claimed is:

1. A process for preparing amorphous ziprasidone mesylate comprising the step of spray-drying a solution of ziprasidone mesylate at an outlet temperature of above 90° C. in a solvent selected from a group consisting of: C₁-C₅ alcohols, C₂-C₈ ethers, glacial acetic acid, optionally in mixture with water.

2. The process of claim 1, wherein the inlet temperature is higher the outlet temperature.

3. The process of claim 1, wherein the solution is formed by dissolving dihydrate needle crystals of ziprasidone mesylate in a solvent.

4. The process of claim 1, wherein the solvent is selected from the group consisting of: diethyl ether, tetrahydrofuran, methyl t-butyl ether, glacial acetic acid, ethanol and mixtures thereof with water.

5. The process of claim 4, wherein the solvent is an ethanol/water mixture.

6. The process of claim 5, wherein the ethanol/water ratio is from about 50:50 to about 95:5 ethanol to water by volume.

7. The process of claim 1, wherein the outlet temperature is about 90° C.

8. The process of claim 7, wherein the inlet temperature is above 90° C.

9. The process of claim 8, wherein the inlet temperature is about 150° C.

10. The process of claim 1, wherein the amorphous ziprasidone mesylate contains less than about 10% of crystalline material as percentage area XRD.

11. The process of claim 10, wherein the amorphous ziprasidone mesylate contains less than about 5% of crystalline material as percentage area XRD.

12. The process of claim 11, wherein the amorphous ziprasidone mesylate contains less than about 1% of crystalline material as percentage area XRD.

13. A process for preparing ziprasidone mesylate crystal form characterized by X-ray powder diffraction peaks at 11.7, 17.3, 23.5, 24.2, and 25.2 degrees two-theta, ±0.2 degrees two-theta (herein defined as Form I) comprising the step of spray-drying a solution of ziprasidone mesylate an outlet temperature of above 70° C. in a solvent selected from a group consisting of: glacial acetic acid, optionally in mixture with C₂-C₈ ethers, and collecting the obtained Form I.

14. The process of claim 13, wherein the inlet temperature is higher than the outlet temperature.

15. The process of claim 13, wherein dihydrate needle crystals of ziprasidone mesylate are used to form the solution.

16. The process of claim 13, wherein the solvent is glacial acetic acid.

17. The process of claim 13, wherein the outlet temperature is from about 70° C. to about 100° C.

18. The process of claim 13, wherein Form I is collected from the upper side of the apparatus.

19. The process of claim 18, wherein Form I is collected from the upper side of the cyclone.

20. A process for preparing ziprasidone mesylate crystal form characterized by X-ray powder diffraction peaks at 17.1, 18.7, 23.8, and 24.4 degrees two-theta, +0.2 degrees two-theta (herein defined as Form VIII) comprising the step of spray-drying a solution of ziprasidone mesylate at an outlet temperature of from about above 45° C. to about 70° C. in a solvent selected from the group consisting of: C₁-C₅ alcohols, optionally in mixture with water.

21. The process of claim 20, wherein the inlet temperature is higher than the outlet temperature.

22. The process of claim 20, wherein the solution is formed by dissolving dihydrate needle crystals of ziprasidone mesylate in a solvent.

23. The process of claim 20, wherein the solvent is a mixture of ethanol and water.

24. The process of claim 23, wherein the ethanol/water ratio is from about 50:50 to about 95:5 ethanol to water by volume.

25. The process of claim 20, wherein the outlet temperature is about 55° C.

26. The process of claim 20, wherein the inlet temperature is above 55° C.

27. The process of claim 26, wherein the inlet temperature is about 80° C.
